(19) United States
(12) Patent Application Publication
(10) Pub. No.: US 2009/0053324 A1
(43) Pub. Date: Feb. 26, 2009

(54) Calcium supplements

(75) Inventors: Dean Neuls, Vancouver (CA); Marcos Neves, Praia Do Canto (BR)

Correspondence Address: NIXON & VANDERHYE, PC, 901 NORTH GLEBE ROAD, 11TH FLOOR, ARLINGTON, VA, 22203, US

(73) Assignee: HL DISTRIBUTION COMPANY, Las Vegas, NV (US)

(21) Appl. No.: 12/289,014

(22) Filed: Oct. 17, 2008

Related U.S. Application Data
(62) Division of application No. 11/298,165, filed on Dec. 8, 2005.
(60) Provisional application No. 60/668,438, filed on Apr. 4, 2005.

(51) Int. Cl. A61K 33/32; A61K 33/06; A61K 33/30; A61P 19/02; A61P 19/10; A61K 33/26

(52) U.S. Cl. 424/639; 424/682; 424/646; 424/641

(57) ABSTRACT

A composition comprising at least 5%, by weight, of calcium and at least 5%, by weight, of magnesium wherein the composition is derived from marine algae.

### PRIORITY INFORMATION

This application claims benefit of priority from U.S. Provisional Application No. 60/668,438 filed Apr. 4, 2005.

### FIELD OF THE INVENTION

This invention relates to calcium compositions. In one preferred aspect, the invention relates to dietary supplements that comprise calcium.

### BACKGROUND OF THE INVENTION

Calcium is an essential mineral and is one of the most popular dietary supplements in the United States. Almost all calcium in the human body is found in the bones and teeth where it is critical for health and strength. Calcium is also found in the blood, extracellular fluid, muscles, and other tissues, where it plays a role in blood pressure, muscle contraction, transmission of nerve impulses, and glandular secretions. Evidence from recent studies suggests that calcium, besides maintaining bone strength, may also be good for the heart, help lower blood pressure, and improve blood lipid levels. An American Cancer Society study also found that calcium may reduce the risk of colorectal cancer.

Experts recommend a daily dose of calcium of 1,000 milligrams for men and premenopausal women (post-menopausal women need 1,200 to 1,500 milligrams). Such a dose is obtainable from dietary sources such as milk, dairy products, and leafy green vegetables but it has been found that the typical American woman gets 625 milligrams and the typical man 865. Therefore, there exists a need for dietary supplements that contain calcium.

Many types of calcium supplements are available in a variety of forms, including tablets, caplets, syrups, chewable tablets, softgels, soft chewable cubes, and antacids. There are also calcium fortified foods and juices. The most common form of calcium used in dietary supplements is calcium carbonate, although there are many different types of calcium available, including calcium malate, calcium citrate malate, calcium lactate, and calcium gluconate. These sources of calcium show varying degrees of bioavailability which may limit their potency. In addition, certain forms of calcium can cause constipation.

In addition to calcium there are many other minerals and other materials that are desirable in a supplement.

One mineral that is useful in a supplement is magnesium. Magnesium is utilized in a variety of essential bodily functions such as ATP metabolism, muscle contraction, nerve function, transmembrane transportation, and enzyme function. Another useful material for a supplement is strontium. It has been suggested that strontium can aid healthy bones.

Many consumers of supplements prefer that the products be derived from ‘natural’ sources. Marine sources are particularly attractive to consumers due to the perceived health benefits. Therefore, it would be advantageous to provide a supplement derived from a marine source that is easily harvested and processed.

### SUMMARY OF THE INVENTION

The present invention relates to calcium compositions. In one preferred aspect the present invention relates to dietary supplement compositions derived from algae, preferably marine algae, that comprise calcium. The present invention also relates to methods of treatment using such compositions. The present invention also relates to methods of processing such compositions. The present invention also relates to kits comprising such compositions.

As used herein, “algae” refers to a non-flowering, stemless, water plant. The term “marine algae” refers to those algae that spend a significant portion of their life cycle in a marine environment.

The present invention is useful for the treatment of a variety of conditions. As used herein, “treatment” means any manner in which the symptoms of a condition are ameliorated or otherwise beneficially altered. Treatment also encompasses prophylaxis. For example, the present invention can be useful for preventing relapse in patients who have previously been cured of the condition.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more.”

### DETAILED DESCRIPTION OF THE INVENTION

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The compositions of the present invention comprise at least 5%, by weight, calcium. Preferably, the present compositions comprise at least 10%, more preferably at least 15%, even more preferably at least 20%, even more preferably still at least 25%, by weight, calcium.

Preferably the compositions herein comprise less than 65%, by weight, more preferably less than 45%, by weight, even more preferably less than 35%, by weight, calcium.

Preferably, at least a portion of the calcium in the present compositions is chelated.

The present compositions preferably comprise magnesium. Preferably, the present compositions comprise at least 1%, more preferably at least 3%, even more preferably at least 5%, even more preferably at least 6%, even more preferably at least 7%, even more preferably at least 8%, by weight, magnesium.

Preferably the compositions herein have weight ratio of calcium to magnesium of less than 5:1, more preferably less than 4:1, and even more preferably less than 3:1.

The present composition preferably comprise one or more vitamins. Preferred vitamins include vitamin C, vitamin D, vitamin K, and combinations thereof

The present compositions preferably comprise carbon. Preferably, the present compositions comprise at least 2%, more preferably at least 5%, even more preferably at least 10%, by weight, carbon.

The present compositions are derived from marine algae. Any suitable marine algae may be used. Preferred are marine algae of the division Rhodophyta (red algae). Preferred are marine algae of the class Rhodophyceae. Preferred are marine algae of the order Corallinale. Preferred are non-geniculate Coralline algae. Preferred are rhodoliths.

Preferably the present compositions comprise other minerals including, but not limited to, chromium, copper, iron, manganese, potassium, strontium, sulfur, zinc, and combinations thereof. It is preferred that the compositions herein comprise strontium. One preferred form of strontium for use herein is strontium citrate. Preferably, the compositions herein comprise at least 25 minerals, more preferably at least 50 minerals, even more preferably at least 70 minerals.

The present composition may comprise other nutritional ingredients. For example, phytonutrients, fatty acids such as such as omega 3, 6, and 9, digestive enzymes such as Betaine HCL, Casein Phospho Peptide (CCP), and combinations thereof.

The compositions herein can be presented in any suitable form including, but not limited to, powders, capsules, tablets, wafers, pastes, gels, liquids, or suspensions.

The present compositions can be prepared in any suitable way. Preferably the compositions of the present invention are manufactured by harvesting the algae, grinding, and sterilizing. It is preferred that the present compositions do not require any reduction of sodium content prior to use. The powder can be encapsulated with a suitable capsule, and packing the capsules in a form suitable for sale to consumers. Preferably the capsules comprise at least 400 mg, more preferably at least 550 mg, of composition.

The compositions herein may be presented in the form of a ‘functional food’. For example, the present compositions can be added to cookies, bread, sports drinks, meal replacement drinks, other soft drinks, and the like. These product presentations can be useful to help subjects who are reluctant to taking a capsule or tablet to ingest the product.

The compositions of the present invention can be used as a dietary supplement. The present compositions are preferably taken on a daily basis. Preferably, at least 1000 mg, more preferably at least 1500 mg, even more preferably at least 2000 mg, of composition is taken daily.

The present invention also relates to kits comprising the composition. The present kit preferably comprises composition as described herein and instructions for ingesting said composition. Preferably the instructions recommend ingestion on a regular, preferably daily, basis. The present kits can comprise other compositions. If present the other compositions preferably comprise strontium, vitamin D, or combinations thereof. Preferably the present kits comprise a composition according to Example 1 and a composition comprising strontium citrate.

The compositions of the present invention can be used to treat a variety of disorders and for a variety of purposes. In particular, the present compositions can be useful in treating osteoporosis, arthritis, bone spurs, plantar fasciitis, or other musculoskeletal disorders, to aid athletic performance, aid recover from injury, improve strength or aid overall physical condition.

The present compositions can be used to aid wound healing or to improve the condition of skin. For example, the present compositions can be used to treat ulcers so as diabetic ulcers or bed sores. Preferably the compositions are applied topically for such uses. For instance, the compositions can be applied to a wound such as an ulcer in the form of a powder, a gel, a paste, or other suitable form. Bandages may also be treated with the present compositions and then applied to the affected area. The present compositions can also be used to treat burns.

The compositions herein are useful for cosmetic applications. They may be used alone or in combination with other ingredients to improve the condition or appearance of the skin.

The compositions of the present invention are useful for treating non-human animals. For example, companion animals such as dogs and cats, working animals such as racehorses and greyhounds, and farm animals such as cattle and sheep may be treated with the present compositions. The present compositions are of particular benefit in helping companion animals suffering from the effects of age or genetic disorders and working animals in improving or maintaining performance.

The present compositions are useful for a variety of other purposes. For example, the present compositions may be used as a pH buffer, in laundry or homecare products, in water filtration, for treating oil or chemical spills, or for improving smell or taste.

### EXAMPLES

It will be understood that the following embodiments of the present invention are intended to be illustrative of some of the possible applications or principles. Various modifications may be made by the skilled person without departing from the true spirit and scope of the invention.

### Example 1

A composition is produced from non-geniculate Coralline algae by harvesting the algae, grinding to a powder, adding 200IU of vitamin D3 and 1.25 mg of vitamin K-2 (menaquinone), encapsulating 600 mg the powder in a Vcap™ capsule, and packing 90 capsules in a bottle, said composition having the following mineral content:

| | PPM | | PPM |
|---|---|---|---|
| Aluminum | 1420 | Mercury | 0.011 |
| Antimony | 4.57 | Molybdenum | <0.1 |
| Arsenic | 0.11 | Neodymium | 3.51 |
| Barium | 5.57 | Nickel | 0.074 |
| Beryllium | 0.081 | Niobium | <0.1 |
| Bismuth | 2.19 | Osmium | <0.05 |
| Boron | 12.1 | Palladium | <0.05 |
| Bromine | 11.5 | Phosphorus | 169 |
| Cadmium | <0.03 | Platinum | <0.05 |
| Calcium | 279,000 | Potassium | 427 |
| Carbon | 124,000 | Praseodymium | 0.63 |
| Cerium | 2.11 | Rhenium | <0.2 |
| Cesium | 2.66 | Rhodium | <0.05 |
| Chloride | 2,370 | Rubidium | 27.6 |
| Chromium | 5.1 | Ruthernium | 0.055 |
| Cobalt | 0.107 | Samarium | 0.67 |
| Copper | 1.59 | Scandium | 0.685 |
| Dysprosium | 1.15 | Selenium | 0.06 |
| Erbium | 18.9 | Silica | 28,000.00 |
| Europium | <0.1 | Silver | 1.78 |
| Fluoride | 7.55 | Sodium | 3970 |
| Gadolinium | 3.04 | Strontium | 2190 |
| Gallium | 1.27 | Sulfur | 940 |
| Germanium | 3.08 | Tantalum | <0.05 |
| Gold | <0.05 | Tellurium | 0.066 |
| Hafnium | <0.1 | Terbium | <0.2 |
| Holmium | <0.1 | Thallium | 0.33 |
| Indium | 0.17 | Thorium | 0.081 |
| Iodine | 10.6 | Thulium | <0.05 |
| Iridium | <0.05 | Tin | 0.197 |
| Iron | 10,100 | Titanium | 31.1 |
| Lanthanum | 0.314 | Tungsten | <0.05 |
| Lead | 0.061 | Vandaium | 12.9 |
| Lithium | 3.9 | Ytterbium | 0.237 |
| Lutetium | 0.31 | Yrrtirum | 1.74 |
| Magnesium | 99,800 | Zinc | 16.2 |
| Manganese | 90.5 | Zirconium | 0.642 |

### Example 2

A composition is produced from non-geniculate Coralline algae by harvesting the algae and grinding to a powder. The composition comprises:

| | Wt. % |
|---|---|
| Calcium | 31.7 |
| Magnesium | 8.3 |
| Carbon | 11.4 |
| Iron | 1.6 |
| Other material | 47.0 |

### Example 3

A kit comprising 90 capsules of a composition according to Example 1 and instructions for ingesting four capsules daily.

### Example 4

A kit comprising 120 capsules of a composition according to Example 1 and 90 capsules comprising 743 mg strontium citrate with instructions for ingesting four capsules daily of the former and three capsules daily of the latter.

### Example 5

A 64 year old female osteoporosis patient loses an average of 1% of her bone mineral density per year. She takes a daily dose of 4 capsules according to Example 1. In addition, she takes 2230 mg per day of strontium citrate. After 10 weeks of following this regimen her physician measures her bone mineral density at her hip by dual X-ray absorptometry and finds it is increased by 5%. Her physician tells her that the bone weakness caused by her osteoporosis is reversing.

### Example 6

A 57 year old male is suffering from acid reflux disease. The patient takes a daily dose of 4 capsules according to Example 1. After 3 months the man reports that he no longer experiences acid reflux even after eating his favorite pepperoni pizza.

### Example 7

A 32 year old male athlete is training for a triathlon. Due to overtraining the athlete has recurrent pain in his left hip and lower back. The athlete takes a daily dose of 2400 mg of a composition according to Example 1 for 4 weeks. The pain in his lower back is completely cured while the pain in his left hip is significantly reduced.

### Example 8

A diabetic 62 year old women has a persistent ulcer on her right leg. She applies 2 g twice a day of a composition according to Example 2 and, after two weeks, finds that the ulcer begins to heal.

### Example 9

A 10 year old male Alsatian dog is diagnosed with osteoarthritis in his left rear hip. 1200 mg of a composition according to Example 2 is given to the dog along with his food. After three weeks the dog shows a marked improvement in his mobility and general condition.

### Example 10

A 5 year old thoroughbred race horse is consistently posting good times on race day. 10 grams daily of a composition according to Example 1 is given to the horse along with his feed. At the following race the horse posts a personal best time.

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. (canceled)

2. The method of claim 10 wherein the composition comprises at least 15%, by weight, calcium.

3. The method of claim 10 wherein the composition comprises at least 25%, by weight, calcium.

4. The method of claim 10 wherein the composition comprises at least 7%, by weight, magnesium.

5. The method of claim 10 wherein the composition comprises one or more vitamins.

6. The method of claim 10 wherein the composition comprises at least one mineral selected from iron, manganese, potassium, strontium, sulfur, zinc, and combinations thereof.

7. The method of claim 10 wherein the marine algae is a rhodolith.

8. The method of claim 10 wherein the marine algae is a non-geniculate coralline algae.

9. The method of claim 10 wherein the weight ratio of calcium to magnesium is less than 5:1.

10. A method of treating osteoporosis, arthritis, bone spurs, plantar fasciitis, or other musculoskeletal disorders by ingesting a composition comprising at least 5%, by weight, of calcium and at least 5%, by weight magnesium wherein the composition is derived from marine algae.

11. A method of treating wounds by topically applying a composition comprising at least 5%, by weight, of calcium and at least 5%, by weight magnesium wherein the composition is derived from marine algae to the affected area.

12. A method of improving skin condition by topically applying a composition comprising at least 5%, by weight, of calcium and at least 5%, by weight magnesium wherein the composition is derived from marine algae to the skin.

13-19. (canceled)

20. A method of treating osteoporosis, arthritis, bone spurs, plantar fasciitis, or other musculoskeletal disorders by ingesting a composition comprising:
(a) at least 5%, by weight, of calcium derived from marine algae,
(b) magnesium,
(c) one or more vitamins.

21. The method of claim 20 wherein the composition comprises at least 25%, by weight, calcium.

22. The method of claim 20 wherein the composition comprises at least 5%, by weight, magnesium.

23. The method of claim 20 wherein the composition comprises vitamin D.

24. The method of claim 20 wherein the weight ratio of calcium to magnesium is less than 5:1.

25. A composition derived from marine algae comprising calcium and magnesium wherein the weight ratio of calcium to magnesium is less than 5:1.
